(19) United States
(12) Patent Application Publication
(10) Pub. No.: US 2019/0279752 A1
(43) Pub. Date: Sep. 12, 2019

(54) GENERATION OF ADHERENCE-IMPROVEMENT PROGRAMS

(71) Applicant: INTERNATIONAL BUSINESS MACHINES CORPORATION, Armonk, NY (US)

(72) Inventors: Edward Vitkin, Nesher (IL); Alex Melament, Kiriat Mozkin (IL); Yardena L. Peres, Haifa (IL); Yevgenia Tsimerman, Haifa (IL); Igor Kostirev-Kronos, Adamit (IL); Pamela A. Nesbitt, Ridgefield, CT (US); Gigi Yuen-Reed, Tampa, FL (US); Navot Naor, Rockville, MD (US)

(21) Appl. No.: 15/917,906

(22) Filed: Mar. 12, 2018

(51) Int. Cl. G16H 20/10; G16H 10/60; G06F 17/30

(52) U.S. Cl. CPC G16H 20/10; G06F 17/30477; G16H 10/60

(57) ABSTRACT

Embodiments of the present invention disclose a method, computer program product, and system for generating medical treatment adherence improvement protocols associated with a target patient. A hierarchical map is received. A query is received to generate an improvement protocol. A patient adherence profile associated with the target patient is generated based on target patient data and corresponding one or more dimensions. An influence value is applied to each corresponding one or more dimensions based on the generated patient adherence profile. A set of dimensions is identified of the corresponding one or more dimensions associated with an influence value crossing a threshold. One or more goals are identifying associated with the target patient. An adherence improvement protocol is generated based on identified one or more goals. User input is received, in response to communicating the generated adherence improvement protocol. The adherence profile, identified goals, and adherence improvement protocol are modified.

### BACKGROUND

The present invention relates generally to the field of medical therapy adherence through data analytics, and more particularly to computer data model assisted generation of an adherence improvement protocol.

A major problem in modern medicine is insufficient adherence to prescribed treatments. This problem is accentuated in chronic disorders, such as Diabetes Mellitus, where the therapy is not curative and must be maintained over a long period or even an entire lifetime. Proper therapy is further exacerbated by treatment regimens being multifaceted and complex, in addition to the disease not having a cure. Treatment regimens may be supplemented with supportive aids in order to assist patients whom otherwise struggle with treatment adherence.

### SUMMARY

Embodiments of the present invention disclose a method, computer program product, and system for generating medical treatment adherence improvement protocols associated with a target patient. A hierarchical map is received, wherein the received hierarchical map includes a plurality of dimensions. A query is received to generate an improvement protocol, based on the received hierarchical map, for a target patient, wherein the query includes target patient data corresponding to one or more dimensions of the plurality of dimensions. A patient adherence profile associated with the target patient is generated based on the target patient data and corresponding one or more dimensions of the plurality of dimensions. An influence value is applied to each corresponding one or more dimensions based on the generated patient adherence profile. A set of dimensions is identified of the corresponding one or more dimensions associated with an influence value crossing a threshold. One or more goals are identifying associated with the target patient, where in the goals are identified based on the set of dimensions of the corresponding one or more dimensions associated with an influence value crossing the threshold. An adherence improvement protocol is generated based on identified one or more goals. User input is received, in response to communicating the generated adherence improvement protocol. The adherence profile is modified based on the received user input. The identified one or more goals are modified based on the modified adherence profile, and the adherence improvement protocol is modified based on modified goals.

### BRIEF DESCRIPTION OF THE DRAWINGS

FIG. 1 is a functional block diagram illustrating a distributed data processing environment, in accordance with an embodiment of the present invention.

FIG. 2 is a functional block diagram illustrating the components of an application within the distributed data processing environment, in accordance with an embodiment of the present invention.

FIG. 3 is a flowchart depicting operational steps of an application, on a server computer within the data processing environment of FIG. 1, in accordance with an embodiment of the present invention.

FIG. 4 is a block diagram depicting a hierarchical map, in accordance with an embodiment of the present invention.

FIG. 5 depicts a block diagram of components of the server computer executing the application, in accordance with an embodiment of the present invention.

### DETAILED DESCRIPTION

Embodiments of the present invention relate to the field of computing, and more particularly to computer data model assisted profile generation of adherence programs. The following described exemplary embodiments provide a system, method, and program product to, among other things, generate personalized adherence improvements programs based on computer model driven factors. Therefore, the present embodiment has the capacity to improve the technical field of adherence improvement protocol generation and medical therapy adherence through data analytics by increasing the efficiency of adherence program generation.

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It may be advantageous to provide patients struggling with a prescribed treatment with a supportive aid. Such support can be provided in different ways, such as professional support (by physicians, nurses, dietitians, social workers, etc), social support (from family and friends) and application-delivered support (by personal smartphones and other devices). The application-delivered support can be either assistant to human support (like forums) or stand-alone (like rule-based applications).

In order to counteract adherence issues in patients with complex medical treatment needs, a personal treatment adherence-improvement program, or adherence improvement protocol may be advantageous. Among other challenges, construction of such program requires profiling of the patient with respect to all known factors and obstacles affecting treatment adherence. Therefore, a definition of comprehensive patient profile including such factors is essential, along with a resulting protocol with the ability to be highly customized.

Currently, there is a significant number of studies analyzing single or very limited number of adherence-affecting factors. However, single factor-based conclusions may have sufficient predictive power to project patient adherence. Therefore, it should be appreciated that it would be advantageous to generate an adherence improvement protocol aggregating all known factors into single adherence protocol.

Such a model does not currently exist for at least a few reasons. First, the appropriate model construction requires a multidisciplinary team with skills in modelling, computer science, healthcare, medicine, psychology, social relationships and others. Second, existing single-factor studies is a non-trivial task due to number of studies on the same factors or even lack of appropriate naming conventions for factors that affect adherence within different research communities. Finally, there is a need for a solution to estimate accurate adherence measurements. Therefore, there is a need for a solution that can ingest data from multiple sources and aggregate factors to generate protocols to aid in the self-management of patients, i.e. providing the event-specific reminders and education, logging the data and generating performance reports to be accessed by patient or health care professional.

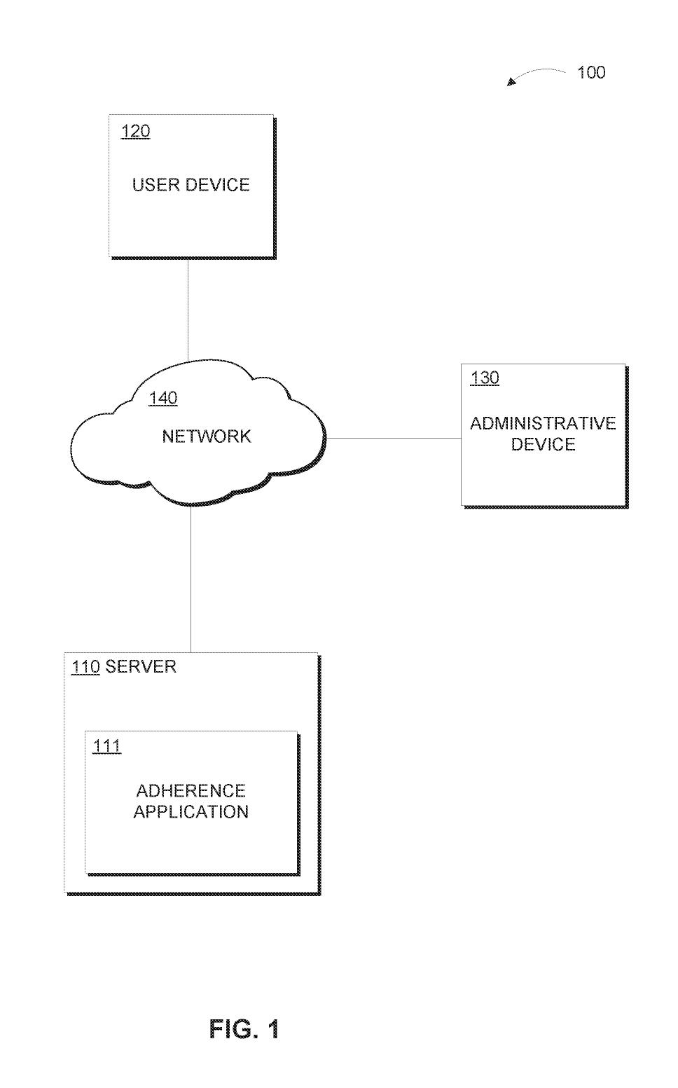

The invention will now be discussed in reference to the Figures. FIG. 1 is a functional block diagram illustrating a distributed data processing environment, generally designated 100, in accordance with an embodiment of the present invention. Distributed data processing environment 100 includes server 110, user device 120, and administrator device 130, all interconnected via network 140.

In various embodiments, network 140 can be, for example, a local area network (LAN), a wide area network (WAN) such as the Internet, or a combination of the two, and can include wired, wireless, or fiber optic connections. In general, network 140 can be any combination of connections and protocols that will support communications between server 110, user device 120, and administrator device 130.

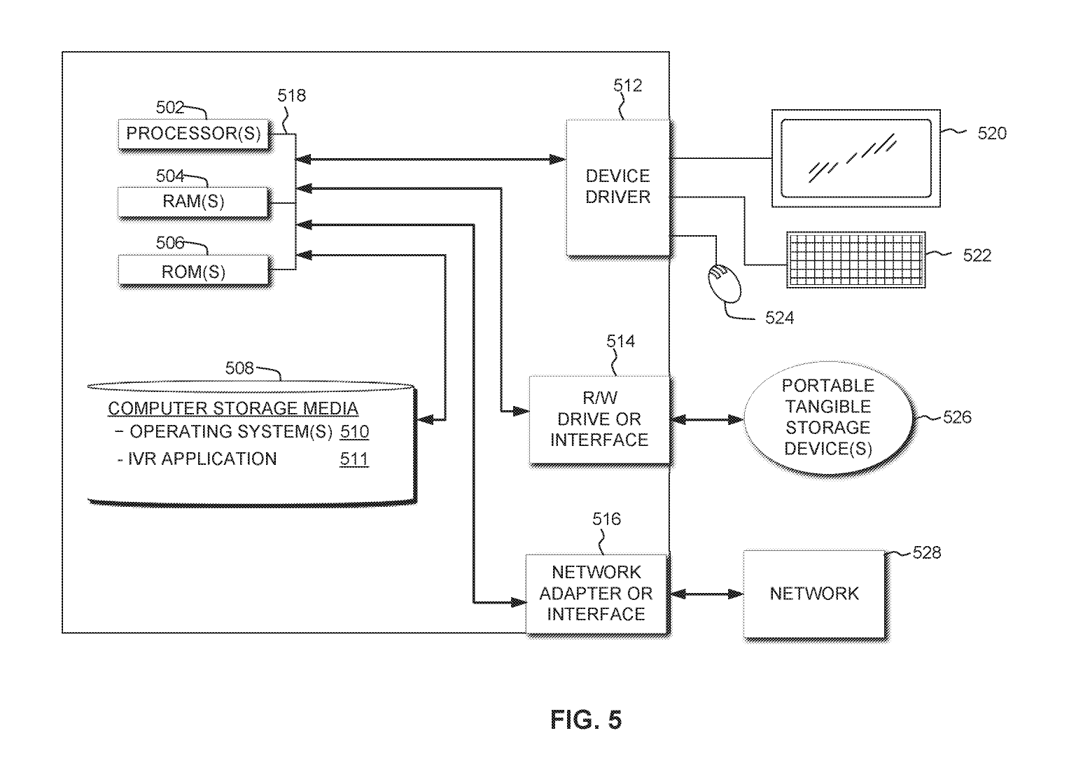

Server 110, user device 120, and administrator device 130 may be a laptop computer, tablet computer, netbook computer, personal computer (PC), a desktop computer, a smart phone, or any programmable electronic device capable of communicating via network, for example, network 140 and with various components and devices within distributed data processing environment 100. Server 110 includes adherence application 111. Product distance application 111 may communicate with user device 120 and product database 130. Server 110, user device 120, and administrative device 130 may include internal and external hardware components, as depicted and described in further detail with respect to FIG. 5.

It should also be noted, that processing for adherence application 111 may be shared amongst server 110, user device 120, and administrative device 130 in any ratio. In various embodiments, adherence application 111 may operate on more than one server, user device, database, or some combination of servers, user devices, and databases, for example, a plurality of user devices 120 communicating across network 140 with a single server 110. In another embodiment, for example, adherence application 111 may operate on a plurality of servers 110 communicating across network 140 with a plurality of user devices 120. Alternatively, adherence application 111 may operate on a network server communicating across the network with server 110 and one or more user devices 120 and administrative devices 130.

In various embodiments, adherence application 111 acts generally to receive a combination of expert feedback (medical healthcare professional), whether prompted or unprompted and various data to generate an adherence improvement protocol for a target patient. In various embodiments, adherence application 111 a target patient is identified and an adherence profile is generated for the target patient. For example, a newly diagnosed patient with Diabetes Mellitus is identified as the target patient and a diabetic treatment adherence profile is generated for the target patient.

In various embodiments, treatment goals are identified and associated with the generated profile by adherence application 111. In various embodiments, goals are identified through publication ingestion or healthcare professional selection input based on a plurality of treatment factors, for example, age, gender, disease type, patient history, identified support system, and various other factors that affect treatment or potential adherence. In various embodiments, treatment factors may be predetermined by a health care professional or data-mined via a machine learning algorithm rom various data sources, for example, historical patient data, publications, patient or health care professional questionnaires, etc.

In various embodiments, adherence application 111, associates a weight or score to each identified goal based on ingested publications and determines the goal or goals associated with a score indicating the most impact based on a weight crossing a threshold or having the highest or lowest value. The score may be associated to each identified goal by any means in the art, for example, ordered lists with lined score values or the like. For example, a target patient profile may contain the patients age and hypoglycemia awareness. The target patients age may have an associated score of −1 and hypoglycemia awareness may have an associated score of −2, therefore, adherence application 111 may identify hypoglycemia awareness as the most negative, or most urgent goal to address. In various embodiments, a plurality of goals may be identified as most urgent.

In various embodiments, adherence application 111 generates an adherence improvement protocol based on one or more of the identified goal or goals. For example, in an embodiment, adherence application 111 generates a protocol including hypoglycemia awareness education on a weekly basis, nutritionist counseling on a weekly basis, and daily blood tests. In various embodiments, adherence application 111 implements the generated adherence improvement protocol by communicating the adherence improvement protocol to a device, for example user device 120, with reminders, appointments, or educational information at a scheduled time.

In various embodiments, adherence application 111 receives user input or device feedback from the target patient via a user device, for example, user device 120, measuring the real-world adherence to the generated adherence improvement protocol. For example, in various embodiments, adherence application 111 presents a adherence questionnaire for display on user device 120 and receives user selection input based on the compliance to the adherence improvement protocol. In additional embodiments, user device 120 receives user input from various sensors on user device 120 with target patient vitals or other data as feedback on the target patient's compliance with the adherence improvement protocol. In various embodiments, adherence application 111 modifies the identified goals, the score associated with the identified goals, and/or the adherence improvement protocol based on the received user input. In various embodiments, where a modification takes place, adherence application 111 may update the adherence profile associated with the target patient.

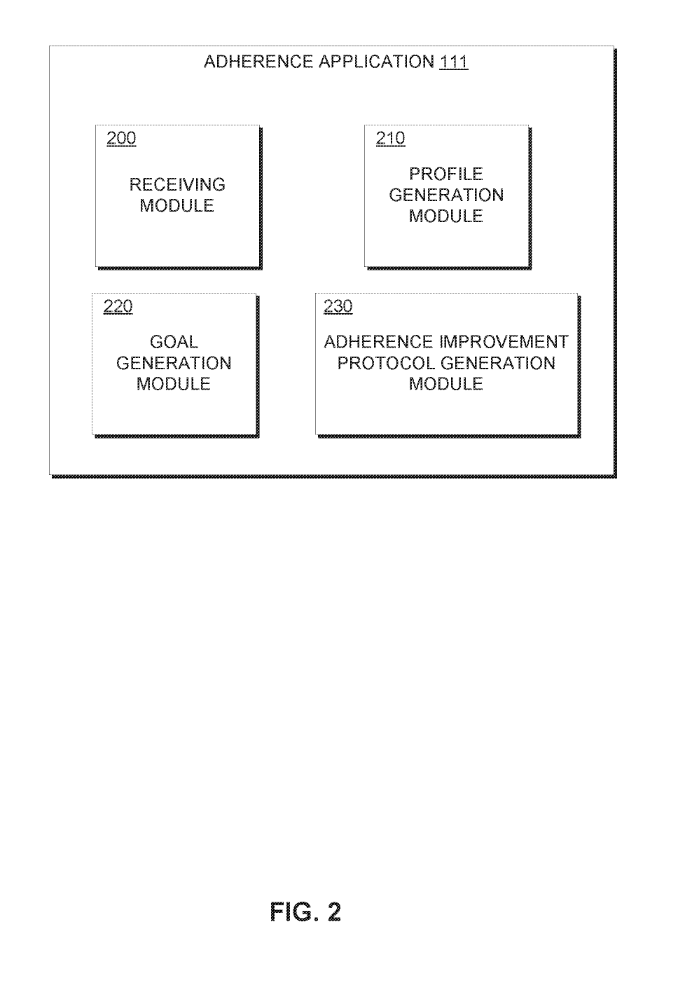

FIG. 2 is a functional block diagram illustrating the components of adherence application 111 within distributed data processing environment 100, in accordance with an embodiment of the present invention. Product distance application 111 includes receiving module 200, profile generation module 210, goal generation module 220, and adherence improvement protocol generation module 230.

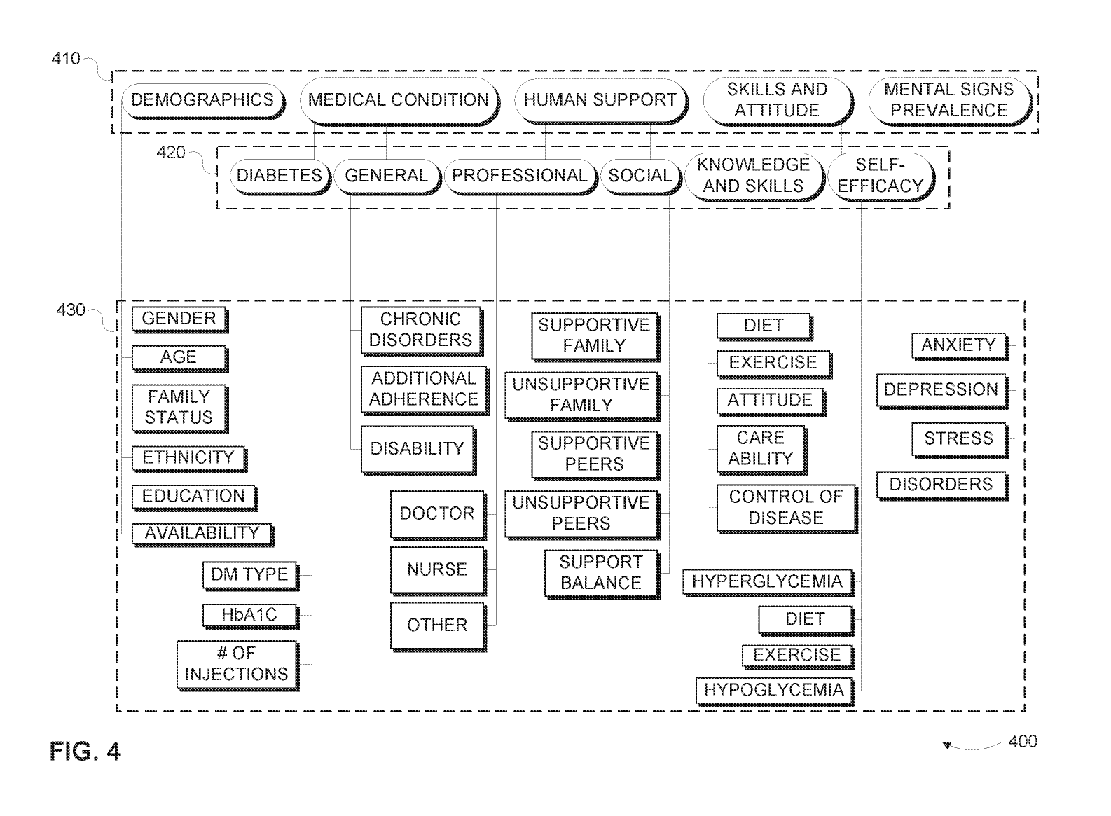

In reference to FIGS. 1 and 2, in various embodiments, receiving module 200 receives target patient identification and a hierarchical map from a device. For example, receiving module 200 may receive a target patient identification from administration device 130 and a hierarchical map, as shown in FIG. 4 from administration device 130. The hierarchical map may be constructed via the ingestion of publications, literature, or protocols related to aspects of treatment-related adherence factors for a disease, class of diseases, or patient populations. For example, a hierarchical map for Diabetes Mellitus may contain factors affecting treatment adherence, for example, demography, mental signs prevalence, human support systems, knowledge, skills and attitude, and medical condition. In various embodiments, the factors may include sub-factors related to the factors. For example, medical condition may include a disease of interest, for example, diabetes mellitus, as one sub-factor, and “other conditions” as a second sub-factor. Each factor or sub-factor may include a plurality of dimensions, where the dimensions are associated with scales or values that are predetermined, provided, or estimated through machine learning. For example, the demography factor may include the dimensions of gender, age, education, etc. where each of the dimensions are associated with a scale or value. For example, the gender value may be “0” or “−1” representing male (0) or female (−1), where analyzed clinical publications suggest that there is a higher mortality rate for patients with diabetes mellitus of one gender when compared to another. In an additional example, age value may be “0” or “−1” representing not elderly or the age of 74 and under (0) or elder or the age of 75 or above (−1), where analyzed clinical publications suggest the mortality rate for patients with diabetes mellitus increases drastically after the age of 75. It should be appreciated by those in the art that the values 1, 0, and −1 are arbitrary and used to represent particular aspects of the corresponding dimensions. In various embodiments, the received hierarchical map is received in tabular data format as shown in Table 1 below:

TABLE 1
Example Hierarchal Table

| Factor | Sub-Factor | Dimension | (1) | (0) | (−1) | (−2) |
|---|---|---|---|---|---|---|
| Demography | | Gender | | M | F | |
| | | Age | | <74 | >75 | |
| | | Family Status | | Single | Not Single | |
| | | Ethnicity | | Caucasian | Other | |
| | | Education | | Normal | Insufficient | |
| | | Availability | High | Normal | Low | None |
| Medical | Diabetes | Diabetes Mellitus | | T1 | T2 | |
| Condition | Mellitus | type | | | | |
| | Diabetes | HbA1C level | | High | Low | |
| | Mellitus | | | | | |
| | Diabetes | Number of prescribed | | Low | High | |
| | Mellitus | insulin injections | | | | |
| | General | Existence of other | | Yes | No | |
| | | chronic medical | | | | |
| | | condition with | | | | |
| | | constant treatment | | | | |
| | General | Adherence to | Good | N/A | Low | |
| | | treatment other | | | | |
| | | chronic medical | | | | |
| | | conditions | | | | |
| | General | Physical disability | | Yes | No | |
| Human Support | Professional | Doctor support | Sufficient | | Insufficient | |
| System | Professional | Nurse support | Sufficient | | Insufficient | |
| | Professional | Other | Sufficient | | Insufficient | |
| | Social | Supportive Family | Good | | Poor | NONE |
| | | behavior | | | | |
| | Social | NonSupportive | Good | | Poor | NONE |
| | | Family behavior | | | | |
| | Social | Supportive Peers | Good | | Poor | NONE |
| | | behavior | | | | |
| | Social | NonSupportive | Good | | Poor | NONE |
| | | Peers behavior | | | | |
| | Social | Support balance | As Needed | | Not Enough/ | |
| | | | | | Too Much | |
| Skills and | Self-Efficacy | Dietary Self- | Good | Normal | Poor | Bad |
| Attitude | | Efficacy | | | | |
| | Self-Efficacy | Hypoglycemia | Good | Normal | Poor | Bad |
| | | Self-Efficacy | | | | |
| | Self-Efficacy | Hyperglycemia | Good | Normal | Poor | Bad |
| | | Self-Efficacy | | | | |
| | Self-Efficacy | Exercise Self- | Good | Normal | Poor | Bad |
| | | Efficacy | | | | |
| | Knowledge | Dietary awareness | | Normal | Poor | Bad |
| | Knowledge | Hypoglycemia | Good | Normal | Poor | Bad |
| | | awareness | | | | |
| | Knowledge | Hyperglycemia | Good | Normal | Poor | Bad |
| | | awareness | | | | |
| | Knowledge | Exercise Barriers | | Normal | High | |
| | | Scale | | | | |
| | Knowledge | Perceived | | Normal | Poor | |
| | | seriousness | | | | |
| | Knowledge | Positive Attitude | | Normal | Poor | |
| | Knowledge | Negative Attitude | | Normal | HIGH | |
| | Knowledge | Care Ability | | Normal | Poor | |
| | Knowledge | Importance of Care | | Normal | Poor | |
| | Knowledge | Self-Care | | Normal | Poor | |
| | | Adherence | | | | |
| | Knowledge | Long-Term Care | | Normal | Poor | |
| | | Benefits | | | | |
| | Knowledge | Control of disease | | Normal | Some | |
| Mental Signs | | Depression signs | | Low | Some | Strong |
| Prevalence | | | | | | |
| Mental Signs | | Anxiety signs | | Low | Some | Strong |
| Prevalence | | | | | | |
| Mental Signs | | Stress signs | | Low | Some | Strong |
| Prevalence | | | | | | |
| Mental Signs | | Signs of Eating | | None | Yes | |
| Prevalence | | Disorders | | | | |

In various embodiments, the values associated with the various dimensions are associated with a positive or negative effect to the patient. In various embodiments, “1” is associated with a positive effect, “0” is associated with a neutral effect, −1 is associate with a negative effect, and −2 is associated with an extremely or severely negative effect based on, in various embodiments, the ingested documentation or health professional feedback.

In various embodiments, values associated with the dimensions for a specific or target patient may be acquired in various sources. For example, receiving module 200 may receive user input from questionnaires, patient databased via integration with hospital systems, from sensors on mobile or wearable devices and/or other devices or methods of data input. In various embodiments, receiving module 200 communicates the received hierarchical map data and target patient data to profile generation module 210.

In various embodiments, profile generation module 210 receives hierarchical map data and target patient data from receiving module 200. In various embodiments, profile generation module 210 generates an adherence profile associated with the target patient identification. For example, profile generation module 210 receives a hierarchical map associated with Diabetes Mellitus and a patient identification number (for example 111) associated with a patient, Patient A, recently diagnosed with Diabetes Mellitus. Profile generation module 210 may generate an adherence profile that includes dimension values based on inputs associated with dimensions received from a device, for example user device 120 via adherence application 111 on server 110. For example, profile generation module 210 may receive Patient A's age as 42, gender as male, Patient A's medical history of having a general practitioner for 5 years, and an answer to a questionnaire that Patient A has never checked blood glucose levels before. Profile generation module 210 may generate an adherence profile that includes Age associated with a 1 value, gender associated with a 0 value, Doctor support associated with a 1 value, and hypoglycemia awareness associated with a value of −2.

In various embodiments, profile generation module 210 periodically queries administrator device 130 for updates in the hierarchical map. In various embodiments, profile generation module 210 receives updated data for the dimensions and/or factors, and in response to receiving updated data, profile generation module 210 modifies, periodically or in real time, the generated adherence profile associated with the target patient based on the received updated data. In various embodiments, profile generation module 210 communicates the generated adherence profile to goal generation module 220.

In various embodiments, goal generation module 220 receives the generated adherence profile from profile generation module 210. In various embodiments, goal generation module 220 utilizes an algorithm in order to identify the most urgent factor that may affect the patient from a clinical and/or efficiency of an improvement in the patient's condition. The most urgent factor, or goal, may be the factor associated with the most negative value in the received adherence profile. In various embodiments, the goal may be identified by a health care professional via a device, for example, administrative device 130.

In various embodiments, goal generation module 220 identifies several factors associated with the most negative value and selects a random factor to be identified as the goal. In various embodiments a factor is selected by goal generation module 220 based on a rule predefined by a user, via a device, for example, user device 120. In various embodiments, goal generation module 220 receives additional data via user input. For example, in various embodiments, receiving module 200 and goal generation module 220 identify a different factor as the goal associated with the target patient. In various embodiments, goal generation module 220 communicates the adherence profile and identified goal to adherence generation module 230.

In various embodiments, a plurality of goals are identified. The number of goals identified by goal generation module 220 may be predetermined by a health care provider input selection via a device, for example, administrative device 130. When the plurality of goals are selected there may be a potential conflict between two goals, for example two goals related to patient education may reduce the uptake of information from individual education topics. In various embodiments, goal generation module 220 determines if any conflicts exist between two or more identified goals and selects one goal of the conflicting goals. In various embodiments, goal generation 220 selects one of the conflicting goals randomly, based on selection input received via server 110, and/or a selection algorithm.

In various embodiments, adherence improvement protocol generation module 230 receives the adherence profile and identified goal(s) from goal generation module 220. In various embodiments, adherence improvement protocol generation module 230 generates an adherence improvement protocol based on the adherence profile and goal(s) associated with the target patient. In various embodiments, the generated adherence improvement protocol is generated for display and adherence improvement protocol generation module 230 receives input selection from a health care professional verifying or modifying the generated adherence improvement protocol In various embodiments, the adherence improvement protocol is predefined by support medical specialists, for example, doctors, nutritionists, fitness experts, psychologists, etc. in order improve one or more goals that most negatively affect adherence. In various embodiments, the adherence improvement protocol is communicated for display to a user device, for example, user device 120, in order to provide the target patient with the adherence improvement protocol to follow. In various embodiments, the use of selection algorithms and health care professional input increases the efficiency of adherence improvement protocol generation and decreases the resource allocation needed in computing power and man hours.

In various embodiments, receiving module 200 receives patience adherence data associate with the Patient adherence is measured from the system-gathered data, such as patient-filled questionnaires, sensor-gathered data via a wearable or mobile device, messages in the system, tracking of patient schedule and the like. For example, if the adherence profile associated with the target patient contains the factor “blood glucose measurements” and the blood glucose measurements was identified as the goal, the adherence improvement protocol may contain a daily requirement for blood glucose measurement. The target patient may input days when blood glucose measurements were taken over the course of a week and a compliance percentage with the adherence improvement protocol may be calculated as a percentage of received blood glucose measurement readings over the amount required by the adherence improvement protocol.

In various embodiments, the adherence profile associated with the target patient, dimensions, factors, goals, and/or the adherence improvement protocol are modified by receiving module 200, profile generation module 210, goal generation module 220, and adherence improvement protocol generation module 230, respectively, based on received patient adherence data.

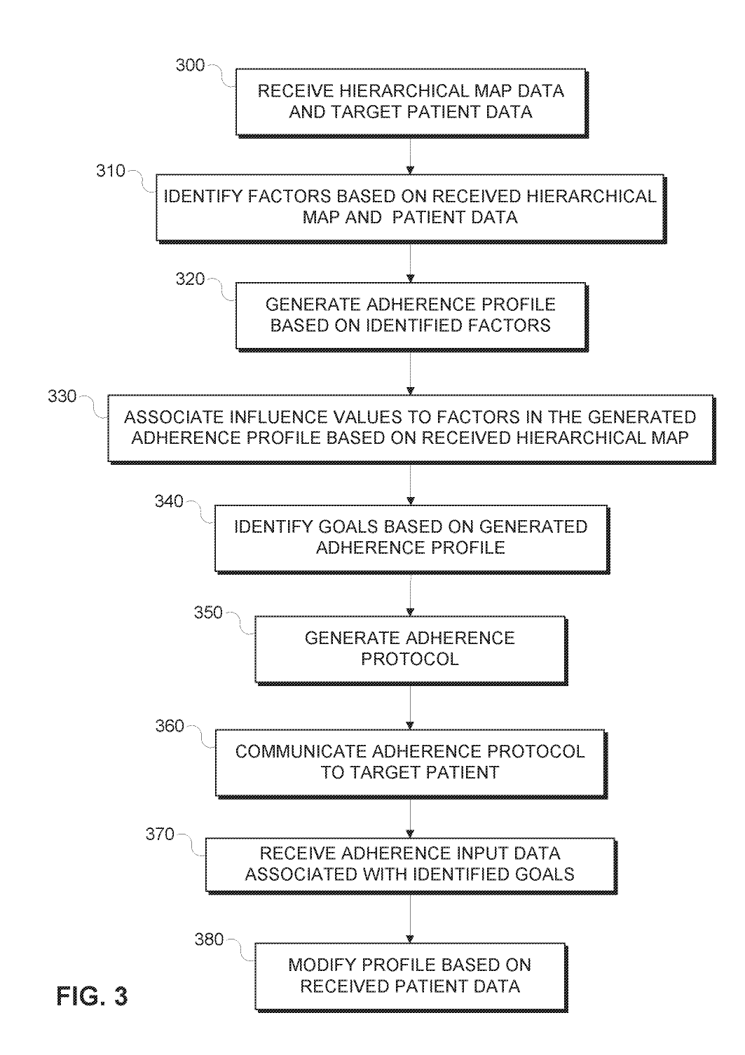

FIG. 3 is a flowchart depicting operational steps of adherence application 111, on server 110 within data processing environment 100 of FIG. 1, in accordance with an embodiment of the present invention.

In various embodiments, receiving module 200 receives a hierarchical map (block 300). The hierarchical map may be received via a device, for example, administrative device 130 from a health care professional. In various embodiments, the received hierarchical map is generated via machine learning by ingesting documentation, for example, patient data and publications and literature related to disease, and categorizing factors that influence a disease path based on the ingested documentation. In various embodiments, a hierarchical map contains categories or factors, sub-factors, and dimensions, as seen in reference to FIG. 4. In various embodiments, receiving module 200 receives a query for the generation of an adherence improvement protocol for a target patient (block 300). In various embodiments, the query includes a target patient identification and target patient data relating to the diagnosis, prognosis, symptoms, and other medical history information associated with the target patient identification.

The target patient may have been recently diagnosed, where the query, for example, from a health care professional, is for an initial adherence improvement protocol, or the query may be associated with a modification to an existing adherence improvement protocol. Receiving module 200 communicates the hierarchical map and associated target patient identification to profile generation module 210.

In various embodiments, profile generation module 210, receives a hierarchical map and target patient query from receiving module 200. In various embodiments, profile generation module 210 indexes the received target patient data and identifies factors and dimensions associated with the target patient based on the patient data and the received hierarchical map data (block 310). For example, if the received hierarchical map includes an “age” factor and the medical history associated with the target patient includes the patients age, profile generation module 210 will identify age as a factor associated with the target patient.

In various embodiments, profile generation module 210 generates an adherence profile associated with the target patient based on the identified factors (block 320). In various embodiments, factors and dimensions have data ranges associated with influence values based on ingested literature or health care professional input. The data ranges are associated with influence values, for example, 1, 0, −1, and −2 representing positive influence, neutral, negative influence and extremely negative influence respectively. In various embodiments, profile generation module 210 associates the factors and dimensions in the generated adherence profile with an influence value based on the received patient data, ranges associated with the factor, and the hierarchical map (block 330). For example, if literature suggests that patients diagnosed with Diabetes Mellitus have a significantly increased mortality rate after the age of 75, the factor of age may have two ranges associated with the factor, ages 74 and below, associated with neutral influence or “0”, and 75 and above associated with negative influence or “−1.” In various embodiments, profile generation module, communicates the generated adherence profile to goal generation module 220.

In various embodiments, goal generation module 220 identifies factors to be labeled goals based on the generated adherence profile (block 340). In various embodiments, factors or dimensions are identified as goals based on being associated with the most negative influence value. If more than one factor or dimension have the same associated most negative influence value a factor or dimension may be identified as the goal at random or according to externally provided rules. In various embodiments, a plurality of goals is identified. The threshold for the number of goals identified may be predetermined or calculated, via machine learning or other algorithms, through statistical analysis of patients' ability to address a maximum number of goals with a required competency. In various embodiments, goal generation module 220 communicates the identified goals and adherence profile to adherence generation module 230.

In various embodiments, adherence improvement program generation module 230 generates an adherence improvement protocol based on the received goals communicated from goal generation module 220 (block 350). The adherence improvement protocol is generated to improve the influence score of the identified goals. For example, if the two goals received by adherence improvement protocol generation module 230 are doctor support insufficient, having an influence score of −1 and control of disease is poor, having an influence score of −1, adherence improvement protocol generation module 230 may generate an adherence improvement protocol including assigning a general practitioner or other healthcare professional to the target patient and a blood glucose reading regime in combination with insulin injection reminders to control the disease. In various embodiments, adherence improvement module generation module 230 communicates the adherence improvement protocol to the target patient, for example, via server 110 to user device 120 (block 360). In various embodiments, adherence improvement protocol generation module 230 communicates the generated adherence improvement protocol to a healthcare professional, for example via server 110 to administrative device 130, and receives user selection input from the health care professional confirming the validity of the adherence improvement protocol or necessary modifications. In various embodiments, adherence improvement protocol generation module 230 modifies the adherence improvement protocol based on the user selection input from the health care professional then communicates the modified adherence improvement protocol to the target patient, for example, via server 110 to user device 120.

In various embodiments, receiving module 200 receives user input, for example via user device 120, that includes adherence data, or data representing the patient's compliance with the adherence improvement protocol (block 370). In various embodiments, the user input is prompted, for example, selection input in response to a questionnaire generated for display, or unprompted, for example, periodic input via sensors on a wearable device associated with the target patient. In various embodiments, receiving module 200 communicates the adherence data to profile generation module 210 and profile generation module 210 modifies the adherence profile associated with the target patient based on the received adherence data (block 380). In various embodiments, a modification in the adherence profile permeates modifications to the factor and dimension influence values, goal identification, and adherence improvement protocol by profile generation module 210, goal generation module 220, and adherence improvement protocol generation module 230, respectively.

FIG. 4 is a block diagram depicting a hierarchical map, generally designated 400, in accordance with an embodiment of the present invention. In various embodiments, factors include demographics, medical condition, human support, skills and attitude, and mental signs prevalence. Sub-factors of medical condition include diabetes and general medical conditions. Sub-factors of human support include professional and social support. Sub-factors of skills and attitude include knowledge and skills and self-efficacy.

Referring to FIG. 4, in an exemplary hierarchical map is depicted, generally designated 400. Exemplary hierarchical map 400 includes factors 410, sub-factors 420, and dimensions 430. In various embodiments, factors 410 include: demography, medical conditions, human support, skills and attitude, and mental signs prevalence. In various embodiments, sub-factors 420 include: diabetes or general medical conditions, professional and social human support, and knowledge and skills, and self-efficacy skills and attitude. In various embodiments, dimensions 430 include: Gender, Age, Family status, Ethnicity, Education, and Availability (under demographics), Diabetes Mellitus Type, HbA1C level, and Number of prescribed insulin injections (under diabetes medical condition), Chronic Disorders, Additional Adherence to other medical conditions, and other disabilities (under general medical conditions), Doctor Support, Nurse Support, and Other Support (under professional human support), Supportive Family behavior, Unsupportive Family behavior, Supportive Peers behavior, Unsupportive Peers behavior, and Support Balance (under social human support), Diet, Exercise, Attitude, Care-Ability, and Control of the Disease (under knowledge and skills), Hyperglycemia, Hypoglycemia, Diet, and Exercise (under self-efficacy), and Anxiety, Depression, Stress, and other Disorders (under mental signs prevalence).

It should be appreciated that in various embodiments, the labels included within factors 410, sub-factor 420, and dimensions 430 are constructed via the ingestion of publications, literature, or protocols related to aspects of treatment-related adherence factors for a disease, class of diseases, or patient populations. In various embodiments, the sources for the ingested data is periodically queried for updates, new data is ingested, and the labels within factors 410, sub-factor 420, and dimensions 430 are modified.

In various embodiments, the functions of adherence application 111 are performed within user device 120 where user device 120 is a wearable or mobile device. In various embodiments, user device 120 is a wearable device that includes biometric sensors, a global positioning system, gyroscopic sensors, an altimeter, a blood oxygenation meter, as well as other sensors to track movement of a user and blood chemistry of a user. In various embodiments, wearable user device 120 receives global positioning system data, gyroscopic sensor data, accelerometer data, altimeter data, blood chemical data, and /or pulse oximeter data, in order to measure or estimate physical activity, medication levels in the blood, or other components in the blood, for example, glucose or insulin levels.

In various embodiments, global positioning system data, gyroscopic data, accelerometer data, altimeter data, and pulse oximeter data allow user device 120 to confirm compliance with physical activity components of the adherence improvement protocol. In various embodiments, blood chemical data, allows user device 120 to confirms compliance with medication or dietary components of the adherence improvement protocol.

The received sensor data allows for automatic adherence data to be received, increasing the efficiency of generating and/or modifying the adherence improvement protocol in real time or at predetermined intervals. This method of adherence improvement protocol modification reduces the computing and human resource cost of gathering adherence improvement protocol data and modifications of the adherence improvement protocol in response to adherence data associated with the target patient.

FIG. 5 depicts a block diagram of components of server 110, user device 120, and administrator device 130 of distributed data processing environment 100 of FIG. 1, in accordance with an embodiment of the present invention. It should be appreciated that FIG. 5 provides only an illustration of one implementation and does not imply any limitations with regard to the environments in which different embodiments may be implemented. Many modifications to the depicted environment may be made.

Server 110, user device 120, and administrator device 130 may include one or more processors 502, one or more computer-readable RAMs 504, one or more computer-readable ROMs 506, one or more computer readable storage media 508, device drivers 512, read/write drive or interface 514, network adapter or interface 516, all interconnected over a communications fabric 518. Communications fabric 518 may be implemented with any architecture designed for passing data and/or control information between processors (such as microprocessors, communications and network processors, etc.), system memory, peripheral devices, and any other hardware components within a system.

One or more operating systems 510, and one or more application programs 511, for example, adherence application 111, are stored on one or more of the computer readable storage media 508 for execution by one or more of the processors 502 via one or more of the respective RAMs 504 (which typically include cache memory). In the illustrated embodiment, each of the computer readable storage media 508 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Server 110, user device 120, and administrator device 130 may also include a R/W drive or interface 514 to read from and write to one or more portable computer readable storage media 526. Application programs 511 on server 110, user device 120, and administrator device 130 may be stored on one or more of the portable computer readable storage media 526, read via the respective R/W drive or interface 514 and loaded into the respective computer readable storage media 508.

Server 110, user device 120, and administrator device 130 may also include a network adapter or interface 516, such as a TCP/IP adapter card or wireless communication adapter (such as a 4G wireless communication adapter using OFDMA technology) for connection to a network 528. Application programs 511 on server 110, user device 120, and administrator device 130 may be downloaded to the computing device from an external computer or external storage device via a network (for example, the Internet, a local area network or other wide area network or wireless network) and network adapter or interface 516. From the network adapter or interface 516, the programs may be loaded onto computer readable storage media 508. The network may comprise copper wires, optical fibers, wireless transmission, routers, firewalls, switches, gateway computers and/or edge servers.

Server 110, user device 120, and administrator device 130 may also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514 and network adapter or interface 516 may comprise hardware and software (stored on computer readable storage media 508 and/or ROM 506).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Based on the foregoing, a computer system, method, and computer program product have been disclosed. However, numerous modifications and substitutions can be made without deviating from the scope of the present invention. Therefore, the present invention has been disclosed by way of example and not limitation.

What is claimed is:

1. A computer executable method for generating medical treatment adherence improvement protocols associated with a target patient, the method comprising:
generating a patient adherence profile associated with the patient based on target patient data in one or more patient dimensions defined by a hierarchical map;
applying an influence value to each of the corresponding one or more dimensions based on the generated patient adherence profile;
identifying a first subset of the one or more patient dimensions exhibiting an influence value crossing a threshold;
identifying one or more goals associated with the target patient based on the first subset; and
generating an adherence improvement protocol based on identified one or more goals.

2. The computer executable method of claim 1 further comprises:
receiving the hierarchical map, wherein the hierarchical map includes one or more factors, wherein each factor of the one or more factors includes one or more hierarchical dimensions;
receiving a query to generate an improvement protocol, based on the received hierarchical map, for the target patient, wherein the query includes target patient data corresponds to one or more patient dimensions of the one or more hierarchical dimensions; and
generating a patient adherence profile associated with the target patient further based on the target patient data and corresponding one or more patient dimensions of the one or more hierarchical dimensions.

3. The computer executable method of claim 1, further comprises:
receiving user input;
modifying the adherence profile based on the received user input;
modifying the identified one or more goals based on the modified adherence profile; and
modifying the adherence improvement protocol based on modified goals.

4. The computer executable method of claim 3, wherein the user input is received in response to communicating the generated adherence improvement protocol.

5. The computer executable method of claim 1 further comprises:
periodically querying a database for updates in the hierarchical map; and
modifying the generated adherence profile in response to receiving updates to the hierarchical map based on the periodic querying.

6. The computer executable method of claim 3 wherein the user input includes adherence data, wherein the adherence data is received via one or more of:
questionnaires;
patient database input; and
remote sensor data.

7. The computer executable method of claim 6, wherein receiving user input further comprises:
receiving adherence data via biometric sensors, and wherein modifying the adherence profile based on the received user input is further based on the adherence data being received via the biometric sensors;
generating goals based on received biometric sensor input; and
periodically monitoring the biometric sensors for additional input.

8. The computer executable method of claim 6, wherein remote sensor data further comprises one or more of:
global positioning system data;
gyroscopic sensor data;
accelerometer data;
altimeter data;
blood chemical level data; and
pulse oximeter data.

9. The computer executable method of claim 1, wherein the threshold corresponds to exiting a range defined by the hierarchical map.

10. The computer executable method of claim 1, wherein the threshold is calculated via machine learning through statistical analysis of the patients' ability to address a maximum number of goals with a required competency.

11. The computer executable method of claim 1, wherein applying an influence value is based on comparing the target patient data to data included in the hierarchical map.

12. A computer program product for generating medical treatment adherence improvement protocols associated with a target patient, the computer program product comprising:
one or more computer-readable storage media and program instructions stored on the one or more computer-readable storage media, the program instructions comprising:
instructions to generate a patient adherence profile associated with the patient based on target patient data in one or more patient dimensions defined by a hierarchical map;
instructions to apply an influence value to each of the corresponding one or more dimensions based on the generated patient adherence profile;
instructions to identify a first subset of the one or more patient dimensions exhibiting an influence value crossing a threshold;
instructions to identify one or more goals associated with the target patient based on the first subset; and
instructions to generate an adherence improvement protocol based on identified one or more goals.

13. The computer program product of claim 12, further comprises:
instructions to receive the hierarchical map, wherein the hierarchical map includes one or more factors, wherein each factor of the one or more factors includes one or more hierarchical dimensions;
instructions to receive a query to generate an improvement protocol, based on the received hierarchical map, for the target patient, wherein the query includes target patient data corresponds to one or more patient dimensions of the one or more hierarchical dimensions; and
instructions to generate a patient adherence profile associated with the target patient further based on the target patient data and corresponding one or more patient dimensions of the one or more hierarchical dimensions.

14. The computer program product of claim 12 further comprises:
instructions to receive user input;
instructions to modify the adherence profile based on the received user input;
instructions to modify the identified one or more goals based on the modified adherence profile; and
instructions to modify the adherence improvement protocol based on modified goals.

15. The computer program product of claim 14, wherein the user input is received in response to communicating the generated adherence improvement protocol.

16. The computer program product of claim 12, wherein receiving user input further comprising:
instructions to periodically query a database for updates in the hierarchical map; and
instructions to modify the generated adherence profile in response to instructions to receive updates to the hierarchical map based on the instructions to periodically query.

17. The computer program product of claim 14, wherein adherence data is received via one or more of:
questionnaires;
patient database input; and
remote sensor data.

18. The computer program product of claim 17, wherein instructions to receive user input further comprises:
instructions to receive adherence data via biometric sensors, and wherein instructions to modify the adherence profile based on the received user input is further based on the adherence data being received via the biometric sensors;
instructions to generate goals based on received biometric sensor input; and
instructions to periodically monitor the biometric sensors for additional input.

19. The computer program product of claim 17, wherein remote sensor data further comprises one or more of:
global positioning system data;
gyroscopic sensor data;
accelerometer data;
altimeter data;
blood chemical level data; and
pulse oximeter data.

20. A computer system for generating medical treatment adherence improvement protocols associated with a target patient, the computer system comprising:
one or more computer processors;
one or more computer-readable storage media;
program instructions stored on the computer-readable storage media for execution by at least one of the one or more processors, the program instructions comprising:
instructions to generate a patient adherence profile associated with the target patient based on a set of received target patient data and corresponding one or more dimensions of a received hierarchical map;
instructions to apply an influence value to each corresponding one or more dimensions based on the generated patient adherence profile;
instructions to identify a set of dimensions of the corresponding one or more dimensions associated with an influence value crossing a threshold;
instructions to identify one or more goals associated with the target patient, where in the goals are identified based on the set of dimensions of the corresponding one or more dimensions associated with an influence value crossing the threshold; and
instructions to generate an adherence improvement protocol based on identified one or more goals.
